(19) United States
(12) Patent Application Publication
(10) Pub. No.: US 2014/0228430 A1
(43) Pub. Date: Aug. 14, 2014

(54) Method of Accelerating Corneal Wound Healing

(71) Applicant: Alcon Research, Ltd., Fort Worth, TX (US)

(72) Inventors: Daniel A. Gamache, Arlington, TX (US); Mark R. Hellberg, Needham, MA (US); Peter G. Klimko, Fort Worth, TX (US); Kerry L. Markwardt, Burleson, TX (US); John M. Yanni, Burleson, TX (US); Eric C. Carlson, Burleson, TX (US)

(73) Assignee: Alcon Research, Ltd., Fort Worth, TX (US)

(21) Appl. No.: 14/156,832

(22) Filed: Jan. 16, 2014

Related U.S. Application Data
(63) Continuation of application No. 13/034,889, filed on Feb. 25, 2011, now abandoned.
(60) Provisional application No. 61/307,947, filed on Feb. 25, 2010.

(51) Int. Cl. C07D 309/30; A61K 9/00; C07C 69/675; C07C 235/06; C07C 59/105

(52) U.S. Cl. CPC C07D 309/30; C07C 235/06; C07C 59/105; C07C 69/675; A61K 9/0048
USPC 514/460; 514/629; 514/557; 514/546

(57) ABSTRACT

The topical ophthalmic use of 5,6,7-trihydroxyheptanoic acid and analogs for the acceleration of corneal wound healing in humans, is disclosed.

The present application is a continuation of U.S. Ser. No. 13/034,889, filed Feb. 25, 2011 and claims the benefit of U.S. Provisional Patent Application Ser. No. 61/307,947, filed on Feb. 25, 2010, both disclosures of which are specifically incorporated by reference herein.

### FIELD OF THE INVENTION

The present invention is directed to compounds and methods for the acceleration of corneal wound healing. In particular embodiments, the present invention is directed to the use of 5,6,7-trihydroxyheptanoic acid and analogs thereof to treat corneal wounds or corneal haze.

### BACKGROUND

Corneal wounds frequently arise from trauma to the eye, such as may occur in automobile accidents, industrial accidents, and wounds caused by weapons. Wounds to the eye also occur as the unavoidable consequence of surgery, such as cataract surgery, penetrating keratoplasty, glaucoma filtering surgery, and refractive surgery such as laser corneal ablation or radial keratotomy. Non-healing corneal ulcers may also arise from pathological non-traumatic causes, such as diabetes or a systemic auto-immune disease such as Sjogren's Syndrome. The healing of these wounds can frequently be slow and difficult, complicating recovery from trauma or the post-operative course of surgery.

Current treatments include the use of topical or systemic anti-inflammatory/immunomodulatory drugs, bandage contact lenses, and autologous serum (Tuli, S. S.; Schultz, G. S.; Downer, D. M. “Science and strategy for preventing and managing corneal ulceration” Ocul. Surf. 2007, 5(1), 23-39). Drug treatments, surgical interventions, and physical methods used for treating corneal ulceration/defective/delayed corneal wound healing frequently are not very effective, have substantial side-effects, or quite inconvenient. For example, use of steroids to treat inflammation-induced ulceration can be complicated by increased IOP, lens opacification side-effects and a reduction in epithelial cell migration (wound closure). The treatment of corneal haze sometimes observed after refractive surgery with the powerful anti-mitotic agent mitomycin C can reduce anterior stromal keratocyte density, possibly compromising the long-term health of the cornea. The compounds of the present invention may have advantages of fewer side effects and/or increased efficacy over these currently employed treatments.

There is, therefore, a need for a readily applicable method of accelerating ophthalmic wound healing, particularly of corneal wounds.

### SUMMARY OF THE INVENTION

The invention provides compositions and methods for the acceleration of corneal wound healing. According to an embodiment of the present invention, a 5,6,7-trihydroxyheptanoic acid or analog thereof is administered to a patient in need of such treatment via topical ocular delivery.

In one aspect, a compound of the invention can be used to treat a corneal wound or corneal haze. In a particular aspect, the corneal wound or haze is the result of diabetes; corneal photoablation due to refractive surgery; chemical burn; inflammation secondary to fungal, viral, or bacterial infection; contact lens wear; traumatic injury; or defects due to: topical medications/preservatives, radiation (including UV light), systemic autoimmune diseases, tear film abnormalities (tear deficiency, lipid or mucin deficiencies); neurotrophic defects; or idiopathic defects.

Specific preferred embodiments of the invention will become evident from the following more detailed description of certain preferred embodiments and the claims.

### BRIEF DESCRIPTION OF THE FIGURES

FIG. 1 shows the effect of 0.1-1% doses of compound 1 on % corneal wound closure in the rat at 24 hours, with comparison to vehicle (PBS=phosphate buffered saline) as negative control and 0.4% hyaluronic acid (HA) as positive control.

FIG. 2 shows the effect of 0.1-1% doses of compound 1 on the number of eyes with complete wound closure in the rat at 24 hours, out of 10 eyes examined PBS vehicle is shown as negative control and 0.4% HA as positive control.

FIG. 3 shows the effect of 0.3-3% doses of compound 5 and 3% compound 1 on % corneal wound closure in the rat at 24 hours, with comparison to PBS vehicle as negative control and 0.4% HA as positive control.

FIG. 4 shows the effect of 0.3-3% doses of compound 5 and 3% compound 1 on the number of eyes with complete wound closure in the rat at 24 hours, out of 10 eyes examined PBS vehicle is shown as negative control and 0.4% HA as positive control.

### DETAILED DESCRIPTION OF THE INVENTION

Unless indicated otherwise, all component amounts are presented on a % (w/v) basis.

Methods and compositions of the invention comprise an ophthalmically acceptable carrier and at least one compound of formula I:

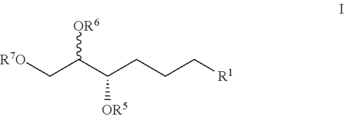

wherein:
R¹ is CO₂R, CONR²R³, or CH₂OR⁴:
R is H, C₁₋₆ straight chain or branched alkyl, C₃₋₆ cycloalkyl, or phenyl, or R¹ is a carboxylate salt of formula CO₂⁻R⁺, where R⁺ is Li⁺, Na⁺, K⁺, or an ammonium moiety of formula ⁺NR⁸R⁹R¹⁰R¹¹;
R², R³ are independently H, C₁₋₆ alkyl, C₃₋₆ cycloalkyl, benzyl, phenyl, OH, OCH₃, or OC₂H₅, provided that at most only one of R², R³ is OH, OCH₃, or OC₂H₅;
R⁴ is H, C(O)R¹², C₁₋₆ alkyl, C₃₋₆ cycloalkyl, benzyl, or phenyl;
R⁵, R⁶, and R⁷ are independently H, CH₃, C₂H₅, C(O)R¹², or CO₂R¹³;
or R⁵ and R⁶ or R⁶ and R⁷ together constitute a carbonyl group (C═O), thus forming a cyclic carbonate;
or OR⁵R¹ together form a cyclic ester (a lactone), as illustrated below

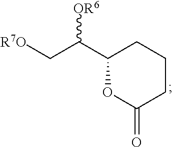

R⁸-R¹¹ are independently H or C₁₋₆ alkyl, each alkyl group optionally bearing an OH or OCH₃ substituent;
R¹² is H, C₁₋₆ alkyl, C₃₋₆ cycloalkyl, benzyl, or phenyl;
R¹³ is C₁₋₆ alkyl, C₃₋₆ cycloalkyl, benzyl, or phenyl; and
[symbol] indicates that the OR⁶ substituent can be arranged to afford the R or S absolute configuration:

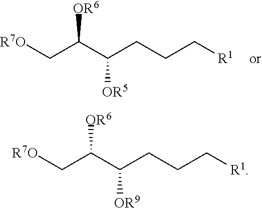

Preferred for use in the methods and ophthalmic compositions of the present invention are those compounds of formula I wherein:
R¹ is CO₂R, CONR²R³, CH₂OR⁴, or a carboxylate salt of formula CO₂⁻R⁺;
R⁺ is Li⁺, Na⁺, K⁺, or NH₄⁺;
R is H, C₁₋₄ alkyl, C₃₋₆ cycloalkyl, phenyl, or benzyl;
one of R² and R³ is H and the other is H, C₁₋₅ alkyl, C₃₋₆ cycloalkyl, benzyl, phenyl, OH, OCH₃, or OC₂H₅;
R⁴ is H, COCH₃, or CH₃;
the absolute stereochemistry at the OR⁶-bearing carbon is as shown below

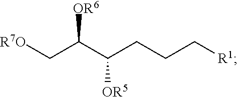

and
R⁵, R⁶, R⁷ are independently H, CH₃, or CH₃CO;
or R⁵ and R⁶ or R⁶ and R⁷ together constitute a carbonyl group (C═O), thus forming a cyclic carbonate;
or OR⁵R¹ together form a cyclic ester (a lactone) as illustrated below

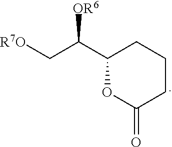

Among the especially preferred compounds for use in the methods and ophthalmic compositions of the present invention are compounds 1-7. Compound 1 is commercially available, for example, from Cayman Chemical Company, Ann Arbor, Mich., and Enzo Life Sciences, Plymouth Meeting, Pa. Compounds 2-7 can be prepared as described in examples 1-6 below.

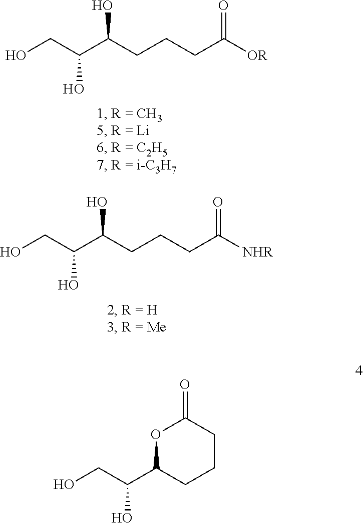

A compound of the invention can be used to treat a corneal wound or corneal haze, including, but not limited to corneal wounds or haze that result from diabetes; corneal photoablation due to refractive surgery; chemical burn; inflammation secondary to fungal, viral, or bacterial infection; contact lens wear; traumatic injury; or defects due to: topical medications/preservatives, radiation (including UV light), systemic autoimmune diseases, tear film abnormalities (tear deficiency, lipid or mucin deficiencies); neurotrophic defects; or idiopathic defects.

In certain embodiments, a compound of formula I is administered in an ophthalmically acceptable carrier for topical ophthalmic administration. The compositions are formulated in accordance with methods known in the art. The compositions may contain more than one compound of formula I. Additionally, the compositions may contain a second drug, other than a compound of formula I.

The compositions of the invention contain an ophthalmically effective amount of a compound of formula I. As used herein, “an ophthalmically effective amount” means an amount sufficient to accelerate corneal wound healing. Generally, the compositions of the present invention will contain from 0.01% to 3% of a compound of formula I. Preferably, the compositions of the present invention will contain from 0.1% to 1% of a compound of formula I.

The compositions administered according to the present invention may also include various other ingredients, including but not limited to surfactants, tonicity agents, buffers, preservatives, co-solvents and viscosity building agents.

Various tonicity agents may be employed to adjust the tonicity of the composition, preferably to that of natural tears for ophthalmic compositions. For example, sodium chloride, potassium chloride, magnesium chloride, calcium chloride, dextrose and/or mannitol may be added to the composition to approximate physiological tonicity. Such an amount of tonicity agent will vary, depending on the particular agent to be added. In general, however, the compositions will have a tonicity agent in an amount sufficient to cause the final composition to have an ophthalmically acceptable osmolality (generally about 150-450 mOsm, preferably 250-350 mOsm).

An appropriate buffer system (e.g., sodium phosphate, sodium acetate, sodium citrate, sodium borate or boric acid) may be added to the compositions to prevent pH drift under storage conditions. The particular concentration will vary, depending on the agent employed. Preferably, however, the buffer will be chosen to maintain a target pH within the range of pH 5.5-8.

Other compounds designed to lubricate, “wet,” approximate the consistency of endogenous tears, aid in natural tear build-up, or otherwise provide temporary relief of dry eye symptoms and conditions upon ocular administration to the eye are known in the art and may be included in the compositions of the present invention. Such compounds may enhance the viscosity of the composition, and include, but are not limited to: monomeric polyols, such as, glycerol, propylene glycol, ethylene glycol; polymeric polyols, such as, polyethylene glycol, hydroxypropylmethyl cellulose (“HPMC”), carboxy methylcellulose sodium, hydroxy propylcellulose (“HPC”), dextrans, such as, dextran 70; water soluble proteins, such as gelatin; and vinyl polymers, such as, polyvinyl alcohol, polyvinylpyrrolidone, povidone and carbomers, such as, carbomer 934P, carbomer 941, carbomer 940, carbomer 974P.

Topical ophthalmic products are typically packaged in multidose form. Preservatives are typically required to prevent microbial contamination during use. Suitable preservatives include: benzalkonium chloride, chlorobutanol, benzododecinium bromide, methyl paraben, propyl paraben, phenylethyl alcohol, edetate disodium, sorbic acid, polyquaternium-1, or other agents known to those skilled in the art. Such preservatives are typically employed at a level of from 0.001 to 1.0% w/v. Unit dose compositions of the present invention will be sterile, but typically will not contain a preservative and will be unpreserved.

A representative eye drop formulation for use in a method of the invention is provided below.

| INGREDIENT | CONCENTRATION (% W/V) |
|---|---|
| Compound of formula I | 0.1-1 |
| Hydroxypropyl methylcellulose | 0.1-0.5 |
| Dextran 70 | 0.1 |
| Sodium Chloride | 0.8 |
| Potassium Chloride | 0.12 |
| Dibasic Sodium Phosphate | 0.025 |
| Edetate Disodium | 0.01 |
| Polyquaternium-1 | 0.001-0.005 |
| NaOH/HCl | q.s. to pH 6-8 |
| Purified Water | q.s. to 100 |

The references cited herein, to the extent that they provide exemplary procedural or other details supplementary to those set forth herein, are specifically incorporated by reference.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Synthesis of Compound 2

A solution of compound 1 in 2 M methanolic ammonia is heated to 85° C. for 1 h in a sealed tube using a microwave reactor. After cooling to room temperature, the solution is evaporated and the residue is purified using silica gel chromatography to afford amide 2.

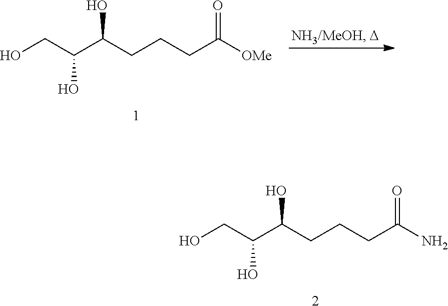 [SMILES: COC(=O)CCC[C@H](O)[C@H](O)CO ; "1" ; NC(=O)CCC[C@H](O)[C@H](O)CO ; "2"]

### Example 2

### Synthesis of Compound 3

A solution of compound 1 in 33% ethanolic methylamine is heated to 85° C. for 1 h in a sealed tube using a microwave reactor. After cooling to room temperature, the solution is evaporated and the residue is purified using silica gel chromatography to afford N-methyl amide 3.

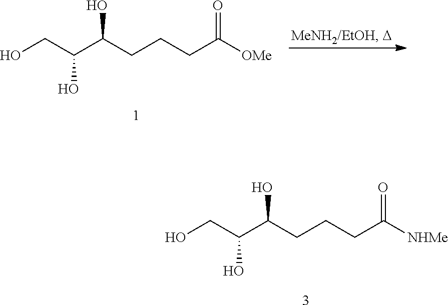 [SMILES: COC(=O)CCC[C@H](O)[C@H](O)CO ; "1" ; CNC(=O)CCC[C@H](O)[C@H](O)CO ; "3"]

### Example 3

### Synthesis of Compound 4

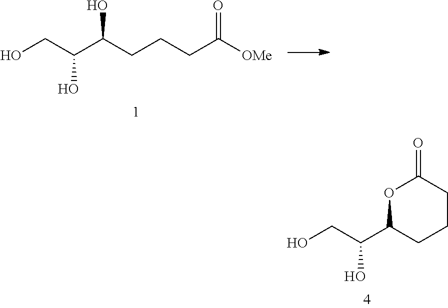

A solution of methyl ester compound 1 (20 mg, 0.104 mmol) in MeOH (2.1 mL) containing 1 M LiOH (0.5 mL, 0.5 mmol) was heated in a microwave heater at 120° C. for 6 minutes. The reaction was concentrated and the residue was chromatographed on a 10 mm diameter×18 cm tall C18 reverse-phase silica gel column eluting with 7:3 v:v 0.05 M HCl:acetonitrile to afford a crude white solid after concentration (40.9 mg). The solid was rinsed with hot CH₃CN (2×2 mL) and the filtrate was concentrated to afford lactone 4 (7.8 mg, 47%). ¹³C NMR (150 MHz, dmso-d₆) δ 171.12 (C), 79.86 (CH), 72.44 (CH), 62.03 (CH₂), 29.39 (CH₂), 21.67 (CH₂), 17.55 (CH₂).

### Example 4

### Synthesis of Compound 5

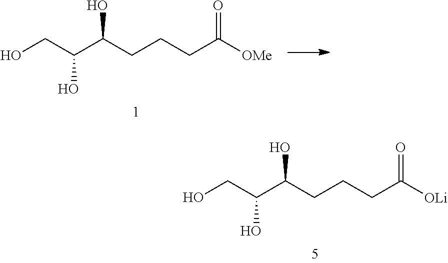

A solution of methyl ester compound 1 in aqueous MeOH is heated to reflux in the presence of 3 equivalents of lithium hydroxide. After 6 h the reaction is cooled to room temperature and the pH of the solution is adjusted to 6 by the addition of 70-9 mesh sulfonic acid resin MP (commercially available from Novabiochem/EMD Biosciences, 10394 Pacific Center Court, San Diego, Calif. 92121). The solution is filtered through a 0.2 μM poly-terfluoroethylene syringe filter and concentrated to afford the lithium carboxylate 5 as a white solid. ¹H NMR (D₂O, 400 MHz) δ 3.69-3.64 (m, 1H), 3.55-3.47 (m, 3H), 2.16-2.12 (m, ²H), 1.67-1.64 (m, 1H), 1.54-1.48 (m, 2H), 1.38-1.34 (m, 1H). ¹³C NMR (D₂O, 100 MHz) δ 183.46 (C), 74.61 (CH), 71.67 (CH), 62.49 (CH₂), 37.26 (CH₂), 31.55 (CH₂), 22.04 (CH₂).

### Example 5

### Synthesis of Compound 6

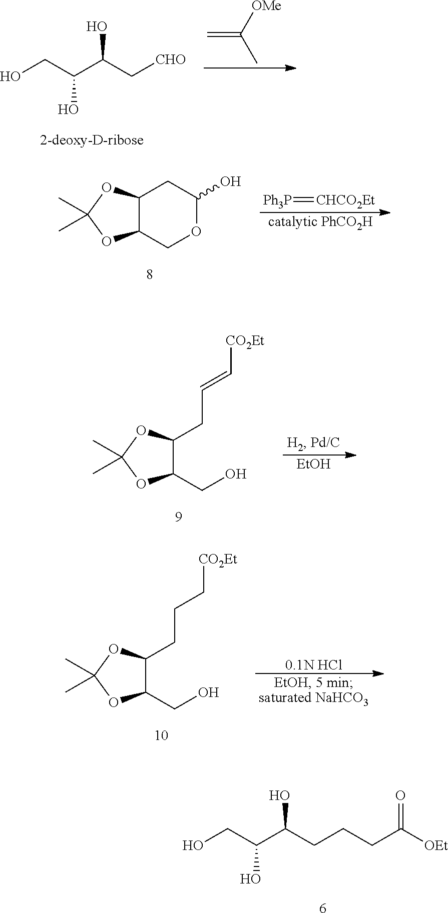

p-Toluenesulfonic acid monohydrate is added to a DMF solution of 2-deoxy-D-ribose, drierite, and 2-methoxy-2-propene to afford lactol 8 after quenching with solid Na₂CO₃ and chromatographic purification. Wittig reaction of 8 with Ph₃P═CHCO₂Et in THF in the presence of catalytic benzoic acid affords enoate 9, which is reduced to 10 under a hydrogen atmosphere in the presence of catalytic Pd/C in ethanol. Deprotection of 10 using 0.1 N HCl in ethanol for 5 minutes, followed by quenching with aqueous NaHCO₃, affords 6 after silica gel chromatographic purification.

### Example 6

### Synthesis of Compound 7

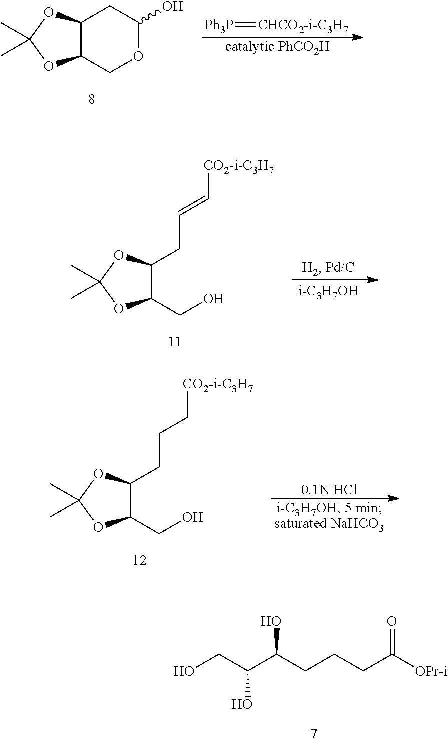

Wittig reaction of lactol 8 (see example 5) with Ph₃P═CHCO₂-i-C₃H₇ in THF in the presence of catalytic benzoic acid affords enoate 11, which is reduced to 12 under a hydrogen atmosphere in the presence of catalytic Pd/C in isopropanol. Deprotection of 12 using 0.1 N HCl in isopropanol for 5 minutes, followed by quenching with aqueous NaHCO₃, affords 7 after silica gel chromatographic purification.

### Example 7

### Efficacy of Compounds 1 and 5 in a Rat Model of Corneal Injury

### Methods

### Corneal Epithelial Wound Generation

Female Lewis rats (175-200 g) were anesthetized with a xylazine/ketamine cocktail (5 mg/kg and 50 mg/kg respectively). A 3 mm diameter circle was superficially demarcated on the central cornea using a 3 mm trephine. Using a corneal rust ring remover (AlgerbrushΠ Alger Co.) tipped with a 1 mm steel burr; the corneal epithelium was removed to the basement membrane within the established 3 mm boundary Immediately following epithelial removal, the corneas were topically stained with 2% sodium fluorescein (Alcon), washed with balanced saline (BSS) and Time 0 hrs images acquired. After 24 hrs, animals were again anesthetized and Time 24 hrs images were acquired.

### Drug Treatments

Immediately after acquisition of Time 0 images, test articles (5 μl) were topically administered to the ocular surface. Animals received topical administration every 2 hrs for the first 8 hrs of the 24 hrs study time. A 0.4% solution of Hyaluronic Acid (HA) was prepared in phosphate-buffered saline (PBS). Compounds 1 and 5 were prepared in PBS and stored at −70° C. until use. During dosing, compounds were stored at 4° C.

### Corneal Fluorescein Staining/Image Analysis

Anesthetized rats (Time 0: xylazine/ketamine; Time 24 hrs: 5% isoflurane gas). The area of the cornea devoid of epithelial cells was stained by topical application of a 2% sodium fluorescein solution (Alcon), followed 10 seconds later with a thorough rinsing with BSS. Images from stained eyes were collected with a Canon digital camera fitted with a macro and close-up lens such that the cornea filled almost the entire image field. A continuous and flash illumination was provided with a Novoflex Cold Light Source Macrolight Plus illuminator. The continuous source was used to focus the eye with the aid of the video output of the Canon camera. A flash lamp illuminator and ring light attached to the camera lens system provided a synchronized and uniform illumination of the corneal surface. Eyes were illuminated and images were captured through a set of filters designed for fluorescein excitation (482±35 nm) and emission (536±40 nm).

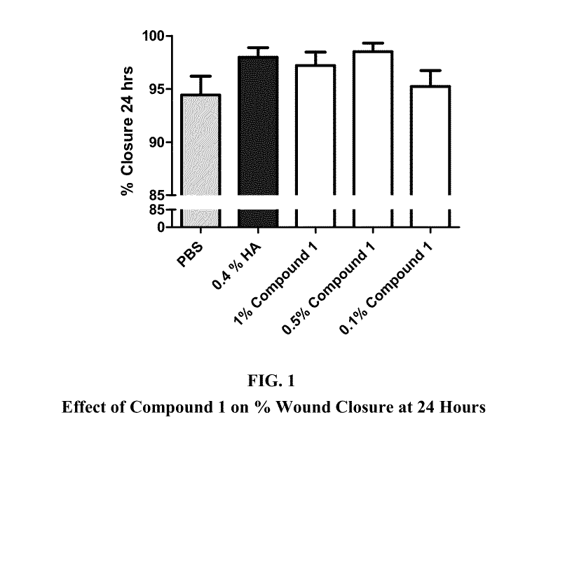

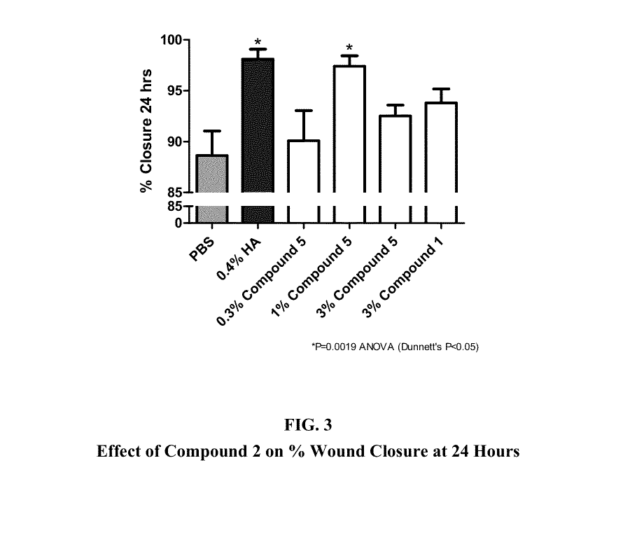

Two image files were recorded for each exposure, a JPG-format RGB (red-green-blue) file used for image display and a Canon Raw file containing the raw sensor data. The raw data was extracted to a 16-bit portable greymap file that was subsequently processed with a demosaic function to make an image of only the green pixels containing the fluorescein information. A circular region of interest (ROI) was centered on the cornea and the automated analysis was performed using RatOSIWound v 2.0 software (Alcon). Automated analysis calculated the total fluorescein positive area of a single large spot indicative of the epithelial wound. The % closure at 24 hours (indicated as “% Closure 24 hours” on the ordinate axis of the graphs in FIGS. 1 and 3) was calculated as

$$\% Closure = \frac{Time 0 - Time 24 hrs}{Time 0} \times 100 ,$$

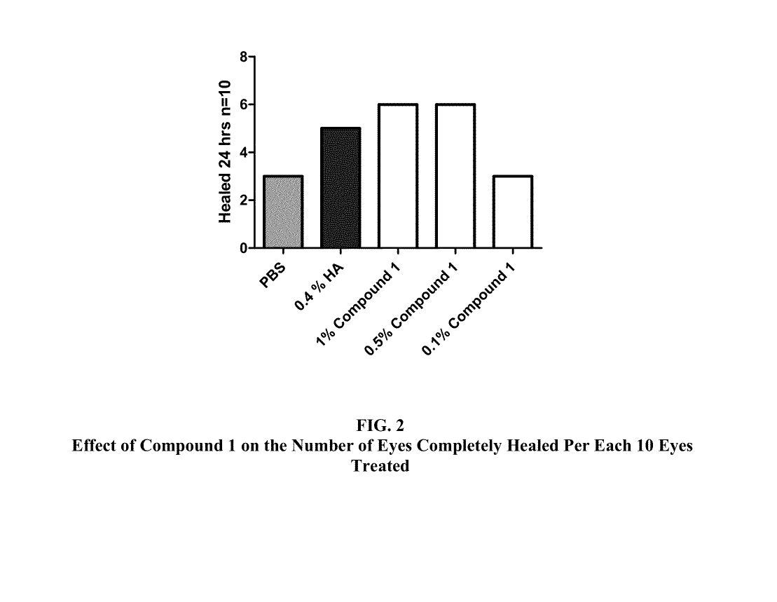

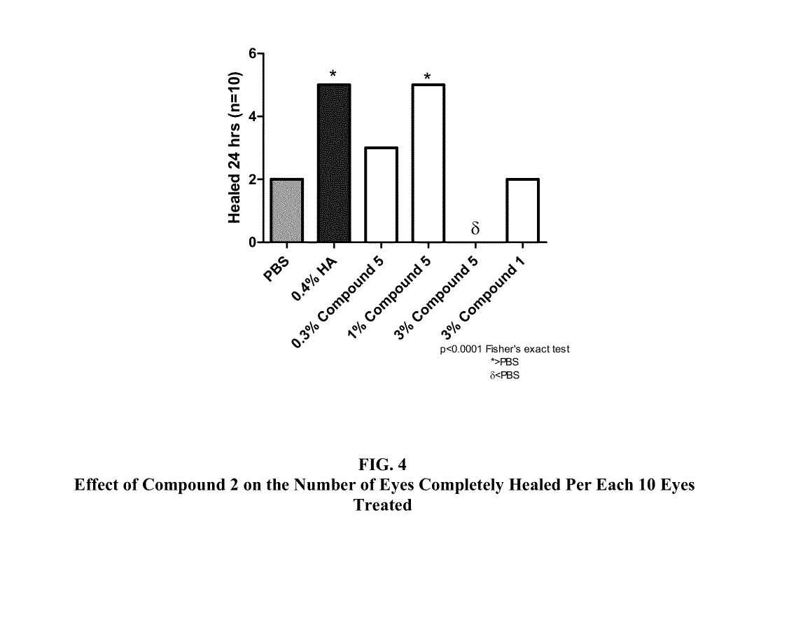

while the number of eyes that were totally healed (that is, had no detectable defect; indicated as “Healed 24 hours n=10” on the ordinate axis of the graphs in FIGS. 2 and 4), normalized as number/10 eyes, was also determined

Data were analyzed using an ANOVA with a Dunnett post-hoc test at a 95% confidence interval.

### Results

Compounds 1 and 5 were evaluated at several doses over the 0.1%-3% w/v concentration range.

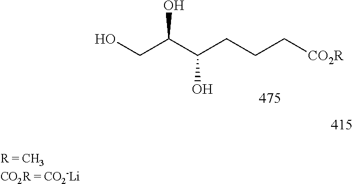

In particular, the 0.5% and 1% doses of compound 1 and the 1% dose of compound 5 were as effective as the positive control 0.4% HA with respect to the % wound closure and the normalized number of completely healed eyes. These data are summarized FIGS. 1-4.

The present invention and its embodiments have been described in detail. However, the scope of the present invention is not intended to be limited to the particular embodiments of any process, manufacture, composition of matter, compounds, means, methods, and/or steps described in the specification. Various modifications, substitutions, and variations can be made to the disclosed material without departing from the spirit and/or essential characteristics of the present invention. Accordingly, one of ordinary skill in the art will readily appreciate from the disclosure that later modifications, substitutions, and/or variations performing substantially the same function or achieving substantially the same result as embodiments described herein may be utilized according to such related embodiments of the present invention. Thus, the following claims are intended to encompass within their scope modifications, substitutions, and variations to processes, manufactures, compositions of matter, compounds, means, methods, and/or steps disclosed herein.

What is claimed is:

1. A method for accelerating corneal wound healing or treating corneal haze in a human needing such treatment, comprising treatment of the affected individual with a topical ophthalmic formulation containing a therapeutically effect amount of least one compound of formula I: 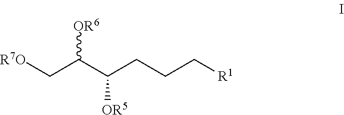
wherein:
R¹ is CO₂R, CONR²R³, or CH₂OR⁴:
R is H, C₁₋₆ straight chain or branched alkyl, C₃₋₆ cycloalkyl, or phenyl, or R¹ is a carboxylate salt of formula CO₂⁻R⁺, where R⁺ is Li⁺, Na⁺, K⁺, or an ammonium moiety of formula ⁺NR⁸R⁹R¹⁰R¹¹;
R², R³ are independently H, C₁₋₆ alkyl, C₃₋₆ cycloalkyl, benzyl, phenyl, OH, OCH₃, or OC₂H₅, provided that at most only one of R², R³ is OH, OCH₃, or OC₂H₅;
R⁴ is H, C(O)R¹², C₁₋₆ alkyl, C₃₋₆ cycloalkyl, benzyl, or phenyl;
R⁵, R⁶, and R⁷ are independently H, CH₃, C₂H₅, C(O)R¹², or CO₂R¹³;
or R⁵ and R⁶ or R⁶ and R⁷ together constitute a carbonyl group (C═O), thus forming a cyclic carbonate;
or OR⁵R¹ together form a cyclic ester (a lactone), as illustrated below 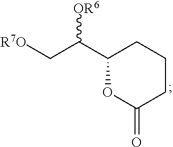
R⁸-R¹¹ are independently H or C₁₋₆ alkyl, each alkyl group optionally bearing an OH or OCH₃ substituent;
R¹² is H, C₁₋₆ alkyl, C₃₋₆ cycloalkyl, benzyl, or phenyl;
R¹³ is C₁₋₆ alkyl, C₃₋₆ cycloalkyl, benzyl, or phenyl; and [symbol]
indicates that the OR⁶ substituent can be arranged to afford the R or S absolute configuration:

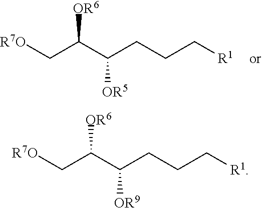

2. The method of claim 1, wherein said treatment comprises treatment with a compound of formula I wherein:
R¹ is CO₂R, CONR²R³, CH₂OR⁴, or a carboxylate salt of formula CO₂⁻R⁺;
R⁺ is Li⁺, Na⁺, K⁺, or NH₄⁺;
R is H, C₁₋₄ alkyl, C₃₋₆ cycloalkyl, phenyl, or benzyl;
one of R² and R³ is H and the other is H, C₁₋₅ alkyl, C₃₋₆ cycloalkyl, benzyl, phenyl, OH, OCH₃, or OC₂H₅;
R⁴ is H, COCH₃, or CH₃;
the absolute stereochemistry at the OR⁶-bearing carbon is as shown below 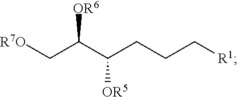
and
R⁵, R⁶, R⁷ are independently H, CH₃, or CH₃CO;
or R⁵ and R⁶ or R⁶ and R⁷ together constitute a carbonyl group (C═O), thus forming a cyclic carbonate;
or OR⁵R¹ together form a cyclic ester (a lactone) as illustrated below

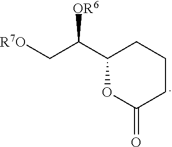

3. The method of claim 2, wherein the compound of formula I is selected from the group consisting of:

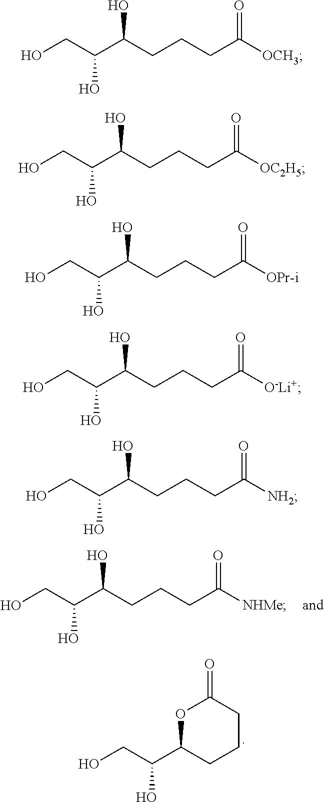

4. The method of claim 1, wherein said corneal wound or haze is the result of diabetes;
corneal photoablation due to refractive surgery; chemical burn; inflammation secondary to fungal, viral, or bacterial infection; contact lens wear; traumatic injury; defects due to topical medications/preservatives; defects due to radiation (including UV light); defects due to systemic autoimmune diseases; defects due to tear film abnormalities; neurotrophic defects; or idiopathic defects.

5. The method of any of claims 1-4, wherein the topical ophthalmic formulation comprises one or more ingredients selected from the group consisting of surfactants; tonicity agents; buffers; preservatives; co-solvents; and viscosity building agents.

6. The method of claim 5, wherein the therapeutically effect amount of a compound of formula I is between 0.01-3%.

7. The method of claim 6, wherein the therapeutically effect amount of a compound of formula I is between 0.1-1%.
